(19) United States
(12) Patent Application Publication
(10) Pub. No.: US 2018/0301211 A1
(43) Pub. Date: Oct. 18, 2018

(54) ELECTRONIC COMMUNITY MEDICAL MARIJUANA NETWORK

(71) Applicants: Christine R. PAPPAS, Reno, NV (US); John KUCHARCZYK, Reno, NV (US); Vu LUU, Las Vegas, NV (US); Mark A. LITMAN, Edina, MN (US)

(72) Inventors: Christine R. PAPPAS, Reno, NV (US); John KUCHARCZYK, Reno, NV (US); Vu LUU, Las Vegas, NV (US); Mark A. LITMAN, Edina, MN (US)

(21) Appl. No.: 15/990,644

(22) Filed: May 27, 2018

Related U.S. Application Data
(63) Continuation-in-part of application No. 15/450,528, filed on Mar. 6, 2017, now abandoned.

(51) Int. Cl. G16H 20/10; A61B 5/00; A61B 5/16; A61B 5/11; A61B 5/024; G16H 50/70

(52) U.S. Cl. CPC G16H 20/10; A61B 5/0022; A61B 5/4848; A61B 5/6801; G16H 50/70; A61B 5/162; A61B 5/11; A61B 5/024; A61B 5/165

(57) ABSTRACT

A system and method for assisting in the use of pain medication comprising:
a) a medical monitoring sensor configured to sense at least one physiological parameter associated with responses to drugs in the human body;
b) a communication network configured to receive and transmit the sensed data relating to the at least one physiological parameter to a processor in communication with the communication network;
c) the processor configured to compare the transmitted sensed data relating to the at least one physiological characteristic to a table stored in memory, the table indicating ranges of acceptable, marginal and unacceptable data;
d) the processor having at least one response for providing at least one medical response to at least one unacceptable range or limit of the transmitted sensed data relating to the at least one physiological parameter; and
i) the processor transmitting at least one medical response to an authorized recipient.

### RELATED APPLICATIONS DATA

This application claims priority as a Continuation-in-Part application under 35 USC 120 from U.S. patent application Ser. No. 15/450,528, filed on 6 Mar. 2017, which in turn claims priority from U.S. Provisional Patent Application Ser. No. 62/379,530 filed 25 Aug. 2016 and titled “ELECTRONIC COMMUNITY MEDICAL MARIJUANA NETWORK”

### BACKGROUND OF THE INVENTION

### 1. Field of the Invention

The present invention relates to the field of integrated electronic community networks and systems of components providing information resources for medical marijuana research and value-based health care services and products.

### 2. Background of the Art

The growing utilization of medical marijuana in health care in the United States is altering the options available to health care consumers and providers. There is evidence that marijuana may be helpful in treating several medical conditions, including: spastic disorders accompanied by pain (including multiple sclerosis, and spinal cord damage); chronic pain (especially neuropathic); nausea (resulting from chemotherapy, radiotherapy, hepatitis C medication, and HIV/AIDS treatment); post-traumatic stress disorder; inflammation; irritable bowel diseases (colitis and Crohn's Disease); and glaucoma.

The United States government classifies marijuana as a Schedule I drug, a category reserved for drugs with no accepted medical use and high potential for abuse. As a Schedule I controlled substance, the federal government mandates that marijuana be evaluated under rigorous clinical trial conditions.

Unfortunately, many of the research findings on marijuana to date have been based on its recreational use. Anecdotal reports are numerous, but relatively few randomized controlled studies have evaluated the risks and benefits of using medical marijuana. A review in Canada identified only 31 studies (23 randomized controlled trials and eight observational studies) specifically focused on medical benefits of the drug. A separate review by the American Medical Association (AMA) also concluded that the clinical research base remains sparse.

One result of the federal prohibition of marijuana has been limited clinical research to investigate the safety and efficacy of marijuana to ameliorate or control symptoms of serious and chronic illness, including chronic pain; nausea; post-traumatic stress disorder; inflammation; irritable bowel diseases; and glaucoma.

There is a need for randomized controlled studies to further evaluate the benefits and risks associated with the use of medical marijuana. In particular, there is a need for controlled human clinical trials that can help establish protocols for marijuana-based treatment of specific medical conditions. Additional research, focused on the benefits and consequences of medical marijuana use for specific disorders, may help to clarify some of these issues.

There is also growing public demand for high quality, value-based health information, research and services to allow individuals to make evidenced-based, clinically appropriate, and medically informed decisions regarding treatment options, including use of medical marijuana.

An electronic medical data aggregator, which ideally is linked to an electronic health record, would enable direct user monitoring, allowing patients to interact with physicians and other trained professionals who can guide them throughout their inpatient and outpatient medical marijuana treatment plan. The primary goal is evidenced-based data yielding quality metrics that a clinician may use in order sets as standard of care for the population in need of higher quality and more cost-effective treatment options.

There is also a need for a disease management program in which medical marijuana health plan beneficiaries can access program clinicians to assess their health needs and advise beneficiaries and their primary care providers of medical marijuana alternative treatment plans. [do not understand this last sentence]. The system would also provide a method for analyzing historical medical provider data to statistically establish a normative utilization profile, and to compare various medical marijuana treatment patterns for a particular diagnosis to determine the highest quality and most cost-effective approach.

There is also a need for a computer-based remote monitoring system for evaluating medical marijuana patients from a remote location to routinely collect information on their health and compliance with their customized and individually prescribed medical marijuana protocols. There is also a need for an effective monitoring system so that clinical studies will be performed under meaningful conditions that can relate to actual conditions of use. Enabling simulation or actual conditions of home use with effective monitoring removes or diminishes the need for personnel being dedicated to a physical presence to take clinical measurements directly. In this way, clinicians and legal authorities can evaluate possible guidelines for appropriate management of the legal use of medical marijuana by investigation within an environment simulating in-home use of marijuana as a medical treatment under legally monitored conditions.

### SUMMARY OF THE INVENTION

One aspect of the invention is to provide health care information regarding various pain management prescriptions, including narcotic pain medications, non-narcotic analgesics, central analgesics, non-steroidal anti-inflammatory drugs, combination analgesics, topical analgesics, and topical anesthetics, as well as medical marijuana and synthetic or natural supplements or drugs that can enable individuals, their caregivers, local health care providers and payers to participate become partners in medical marijuana-based health care management.

Another aspect of the invention is to enhance patient management and continuity of patient care through electronic exchange of specific, timely, and secure patient specific information regarding such pain management prescriptions, including medical marijuana.

A further aspect of the invention is to provide for a health industry development program focused on such pain management prescriptions, including, medical marijuana, including government, health care providers and payers, the clinical and information technology industry, and the community involved in the management of health care.

A further aspect of the invention is to enable legal jurisdictions in conjunction with established medical practitioners to evaluate the potential performance and benefits of such pain management prescriptions, including medical marijuana, under user dominated conditions.

Another aspect of the present invention is to provide an effective information delivery system for such pain management prescriptions, including medical marijuana, which supports a range of mediums, including personal and network computers, multimedia kiosks and smart card technology, cable and pay television, cable and pay television, television, fax and traditional mail.

Another aspect of the present invention is the ability to track timing, frequency, and quantity of drug usage during dosing of such pain management prescriptions, including medical marijuana. Precise dosing information is currently not available and through the tracking ability of the present invention, it will be possible to build a database of usage patterns to evaluate safety and efficacy associated with specific usage patterns.

A further aspect of the present invention is to ensure that all information being recorded is protected, encrypted, and secure, acknowledging that the vital information being collected is protected healthcare information.

The invention relates to an integrated electronic community medical marijuana health care network, with a personal health care system, a networked health care and monitoring system, and a software-based medical information system. The personal health care system comprises a health monitoring device executing a method that is linked with a computer system for receiving, storing, processing, and transmitting information related to the use of pain management prescriptions, including medical marijuana, by patients, including medical marijuana patients, utilizing a plurality of interfacing ports. In conjunction with the personal health care system of the present invention, a networked health care and monitoring system is provided which is capable of providing updated reliable vital information on the health condition of such pain management prescription consumers, including medical marijuana consumers, to health care providers and payers. In particular, the networked health care and monitoring system is adapted to support home health care and maintenance of such pain management patients and their prescriptions, including medical marijuana patients. In conjunction with the personal health care system and the networked health care and monitoring system of the present invention, a software-based medical information system performs a method of analyzing health care claims records for an enrolled population of medical marijuana consumers to assess and report on quality of health care based on established quality indicators.

A personal health care system is designed to evaluate the safety and efficacious use of pain management prescriptions, including medical marijuana prescriptions to medical marijuana patients, by providing:

a) a wearable health monitoring sensor configured for contact with the human body;

b) the health monitoring sensor configured to sense, register and analyze continuously or by preset and programmed periodicity at least one marker or parameter associated with psychological, physiological or metabolic responses to medical marijuana in the human body;

c) the health monitoring sensor configured to either transmit continuously or periodically or store (for later transmission) sensed data relating to the at least one psychological, physiological or metabolic marker or parameter;

d) a communication network configured to receive and transmit the sensed data relating to the at least one psychological, physiological or metabolic marker or parameter either by hardwired or wireless communication with the sensor or by physical reception of a memory storage device containing the stored sensed data relating to the at least one psychological, physiological or metabolic marker or parameter;

e) a processor in communication with the communication network and configured to receive and store in memory transmitted sensed data relating to the at least one psychological, physiological or metabolic marker or parameter;

f) the processor configured to compare the transmitted sensed data relating to the at least one psychological, physiological or metabolic marker or parameter to a reference chart stored in memory, the reference chart indicating ranges of normal, borderline and abnormal values with respect to the transmitted sensed data relating to the at least one psychological, physiological or metabolic marker or parameter;

g) the processor having at least one response in memory for providing at least one corrective health response to at least one abnormal value of the transmitted sensed data relating to the at least one psychological, physiological or metabolic marker or parameter; and

h) the processor configured to transmit at least one corrective health response to an authorized recipient of that at least one corrective health response.

Wearable health monitoring devices rely on body-generated data to monitor the current state of psychological physiological and metabolic parameters and record the data for subsequent analysis. The individual wearing the device, their physician, and other authorized individuals can get real-time collected data for analysis before recommending a course of action for treatment.

These moderately automatic or patient controlled monitoring sensors may be combined with or used in parallel with more invasive testing, such as non-wearable health monitors, including finger oximeters, sleep oximeters, sleep monitors, apnea monitors, as well as physician administered testing such as DNA tests, RNA tests and telomere testing functional and code tests.

Pain management treatments are defined within the scope of the present technology as any application via intravenous or intra-arterial injection, oral or gastrointestinal delivery, or other methodology of introducing drugs into a patient's system. Drugs are any chemical agent used in the prevention, diagnosis, treatment or cure of disease or disorder, including substances, such as cannabis and narcotics, that are of concern for abuse, misuse, causation of harmful side effects, or any other condition that warrants patient monitoring during initial or prolonged use.

### BRIEF DESCRIPTION OF THE DRAWINGS

FIG. 1 is a schematic of a monitoring and response system according to the present invention. The system comprises wearable sensors, signal registration and transmission circuitry, supply unit, and other components. The present invention uses novel technology to create an intelligent and flexible platform for biometric healthcare analytics. The hardware and software systems of the invention are designed to maximize health data inputs. Whereas traditional electronic devices are rigid, bulky and fundamentally mismatched to the properties of the human body, the present invention provides thin and flexible products that accommodate the movements of the individual wearer. The software platform according to the present invention consists of a complete end-to-end system with mobile interfaces, cloud storage and analytical tools. The software allows for the creation of robust systems to support the high volume of data gathered by the health monitor and analytic platforms and machine learning.

FIG. 2 is a flow-chart illustrating a process for monitoring and responding to sensed psychological, physiological or metabolic markers or parameters occurring during the use of medical marijuana by a medical marijuana consumer.

FIG. 3 is a flow-chart illustrating a method for monitoring, registering and analyzing the sensed psychological, physiological and metabolic markers or parameters according to the invention. Statistical methods are employed to check the significance of changes in the sensed psychological, physiological, and metabolic markers or parameters.

FIG. 4 shows a diagram representing various health influences related to the use of home treatments and medical marijuana treatments.

### DETAILED DESCRIPTION OF THE INVENTION

A system is designed to monitor and respond to psychological, physiological or metabolic events that can occur in patients using pain management prescriptions or during the use of medical marijuana. The discussion in the remainder of the text will emphasize medical marijuana for convenience and economy of style, although it should be interpreted as including any and all pain management prescriptions and other materials introduced to a patient that are being managed. The system can operate by providing:

a) a wearable or applicable (contacting or directed sensing) health monitoring sensor configured for contact or functional access to the human body. The sensor may be a dedicated device such as a band-supported monitor attached to the arms, legs, chest, waist, neck or head, and non-limiting examples of structures may be in the form of a head band, a watch, a scarf, a wrap, a leg band, an arm band, a wrist band and the like, and their appearance may be made to look like common commercial and every day articles for apparel or entertainment, such as iPods, phones with jacks, health belts, eye wear and the like. The watches, wristbands, gloves and the like that interact with the system are similar to commercial equipment used in gaming devices which can interpret both gross and subtle movements. They may be further adapted with pulse monitors, temperature monitors, heart beat monitors, force sensors (especially on the fingers and palms) so as to enable input of the further requested data from sensing. Optical focus sensors (eyeglass devices that sense the directional and distance focus of the human eye) can also be used in the sensing. This eyewear is transparent, so vision of the user is not impaired, but can still detect the objective focus parameters of the user. This can be important in determining objective physiological capability during testing by determining optical response times to changes in a visual screen during performance of tests, identifying response times affected by treatment. The movement elements would have at least three-directional attuned accelerometers providing information through wireless communication to the specific client processor. They may also be individual commercial devices, used collectively, separately or contemporaneously to provide individual sensed data components. The contact (or where possible, with directed focus at and reception from a human body) with the human body need not be the source of the sensing, as with eyeglasses. The eyeglasses may contain light sensors which can receive light reflected off the eyeball to determine pupil dilation. Separate sensors may be on each lens or each frame around the eyes.

b) the health monitoring sensor configured to sense at least one marker or parameter associated with a psychological response to marijuana in the human body. Among the non-limiting indices or parameters that can be sensed in this regard are at least one or more indices or correlations with psychological data scales, such as the Pain Scale, Quality of Life Scale, Mood Scale, and various functional scales, which provide an overall personalized psychological health perspective of the medical marijuana patient, which non-objective markers would be supplemented by direct patient input into the system through a keypad or voice reception (with translation into digital or written input), as well as by direct clinical observation of the medical marijuana patient.

c) the wearable or applicable health monitoring sensor configured to sense at least one marker or parameter associated with a physiologic response to marijuana in the human body. Among the non-limiting markers or parameters that can be sensed in this regard are at least one or more of pulse rate, heart rate, electrocardiogram, systolic blood pressure, diastolic blood pressure, pulse pressure, mean arterial blood pressure, change in blood pressure, core body temperature, peripheral body temperature, change in core body temperature, change in peripheral body temperature, respiration rate, respiration flow rate, skin temperature, perspiration rate (especially at specific ambient temperature and physical activity or inactivity), blood oxygenation levels, blood sugar levels, muscle tremors, mouth dryness, physical balance control, skin color, shivering, and the like.

d) the wearable health monitoring sensor configured to sense at least one marker or parameter associated with a metabolic response to marijuana in the human body. Among the non-limiting markers or parameters that can be sensed in this regard are at least one or more of body weight and body mass index, blood pressure, fasting plasma glucose, fasting lipid profile, skin temperature and respiration rate, and the like.

e) the wearable health sensor is configured to either transmit (immediately or upon a timed or signaled delay at the end of specific events or time periods) or store sensed data relating to the at least one psychological, physiological, or metabolic marker or parameter. The sensed data may be stored in an erasable memory on the device itself, the device itself may have or be in communication with a data storage device (e.g., flash drive, disk, hard drive, tape, and the like) which can then be downloaded onto a portable memory device, and can be transmitted to or carried to the computer/processor (generically referred to herein as a main frame computer or other computing device) which is a core element in the operation of this system.

f) a communication network is configured to receive and transmit the sensed data relating to the at least one psychological, physiological, or metabolic marker or parameter either by hardwired or wireless communication with the sensor or physical receipt of a memory storage device containing the stored sensed data relating to the at least one health marker or parameter. The communication network may be a wireless system directly or indirectly from the health monitoring device, from the memory associated with and receiving sensed data from the health monitoring device and may include a port into which permanent memory may be transmitted or a temporary memory device (e.g., flash drive) may be inserted (e.g., a USB port) to transfer from the memory device or the original sensed data source to the computer/processor.

g) a processor in communication with the communication network and configured to receive and store in memory transmitted sensed data relating to the at least one health marker or parameter. The data may be received and stored as raw data or raw signals and converted by software into actual values that may be displayed, printed and read by a user. For example, blood pressure data may be originally transmitted in digital form, wherein the processor receives and converts the raw digital data into displayable and readable data such as 120/80. Alternatively, the sensor may itself be capable of transmitting the data as readable, displayable and printable information by having a microprocessor or digital-to-analog converting component (e.g., filed programmable gated array—FPGA, or application specific integrated circuit—ASIC) in the sensor or between the sensor and the computer.

h) the processor is configured to compare the transmitted sensed data relating to the at least one health marker or parameter to a reference table stored in memory, the table indicating ranges of normal, borderline and abnormal values with respect to the transmitted sensed data relating to the at least one health parameter. The reference tables can be standard tables available in a generically programmed computer for the system, and/or may be a reference table or tables configured for specific patients or for specific types of patients. For example, the reference table for responses to blood pressure readings may have a general table for people who entered the program with a normal, pharmacologically-untreated blood pressure reading, and a separate reference table or specifically physician adjusted table based on identified controls, which may be more or less inclusive with respect to normal values for that particular patient cadre or similar groups of patients.

h) the processor having at least one response in memory for providing an at least one corrective response to at least one abnormal value of the transmitted sensed data relating to the at least one health marker or parameter. The responses can include direct changes in the quantity of medical marijuana, time intervals and types of marijuana that can be used, as well as physician prescribed changes in existing medications of record, or even an alert for the medical marijuana patients to consult with or immediately visit the primary physician or specialist.

i) the processor configured to transmit the at least one health response to an authorized recipient of that at least one health response. The transmission will include the medical marijuana patient, either through the network itself or to a patient telephone number, e-mail address or other contact point, and the message, especially where a significant health response is being suggested, to the appropriate physicians or other caregivers identified in the system, including at least the prescribing physician and the specialist for a condition indicated as material to the identified issues requiring intervention with medical marijuana.

The term “applicable” is used to differentiate from wearable in that the applicable device need not be designed to be actually worn, but may be a patch, wand, tube, adhered sensor, or directed system (e.g., infrared emitter and infrared reader of reflected/diminished infrared radiation). The term applicable is therefore broader than “wearable” as it includes wearable and non-wearable sensors. Where the term “wearable” is used herein, it is exemplary and is not exclusive of applicable, but is evidence of one format of applicable.

These moderately automatic or patient controlled monitoring sensors may be combined with more invasive, Physician or clinician administered testing such as DNA tests, RNA tests and telomere testing functional and code tests. Pain management treatments are defined within the scope of the present technology as any application, introduction, injection, including intravenous and intra-arterial delivery, oral and gastrointestinal delivery, or other delivery methodology of providing drugs into a patient's system. Drugs are any chemical agent used in the prevention, diagnosis, treatment or cure of disease or disorder, including substances, such as cannabis and narcotics, that are of concern for abuse, misuse, causation of harmful side effects, or any other condition that warrants patient monitoring during initial or prolonged use.

The system may have the health monitoring sensor as an electronic device configured to be wearable or at least temporarily secured to a human body during a sensing period of time. The sensor is preferably a non-invasive sensor which does not puncture the skin of the medical marijuana patient, although an implanted sensor can be used in certain situations where critical psychological, physiological, or metabolic information cannot be obtained by external non-invasive means. The health monitoring sensor senses for at least one health marker or parameter selected from the non-limiting health categories comprised of cardiovascular markers or parameters, respiratory markers or parameters, and neurocognitive markers or parameters. Respiratory and cardiac markers or parameters can be obtained, for example, using respiratory inductive plethysmography embedded in a chest band sensor. Skin conductance measurements can be obtained using electrodes in a chest band, head band, or arm band. Skin temperature can be obtained with surface probe thermistors. Multiple-axis accelerometers can be used for motion sensing. Body temperature, can be monitored with a sensor that employs thermocouples or thermistors that are sensitive to conductive and convective heat transfer. Alternatively, temperatures can also be measured by non-contact sensors that are sensitive to infrared radiation. Infrared thermometers that operate in the physiological temperature range are readily available commercially. In the method of the invention, more sophisticated skin surface temperature measurements are also possible by incorporating an infrared thermometer into a remote scanner so that the entire body, or a regional portion of the body, is scanned and a thermal image is produced to give a quick indication of the relative temperature in the scanned body region. Further in the method of the invention, heart rate and related parameters can be monitored via a capacitance skin electrode sensor that functions as an electronic stethoscope to provide for an electronic communication with a remote ECG monitor. To some degree, blood chemistry can be evaluated by transcutaneous transmission and reception of electromagnetic radiation. Examples of such technology are provided in U.S. Pat. Nos. 5,433,197; 6,124,134 and 7,397,566 (Stark)

Further in the method of the invention, a single sensor may be able to monitor multiple parameters, as for example, a single sensor can read both blood pressure and temperature with only slight adjustments in software or the provision of multiple sensing chips within a single device. The multiple reads can be simultaneous and/or sequential. The sensor may be configured to emit electromagnetic radiation and receive returned electromagnetic radiation and provide data with respect to amounts of difference in energy levels or characteristics of received electromagnetic radiation as compared to emitted electromagnetic radiation. The sensor may be configured to sense audio, visual, pressure or electromagnetic information and convert the information into electromagnetic signals that are stored or transmitted, for example, wirelessly to a smart phone.

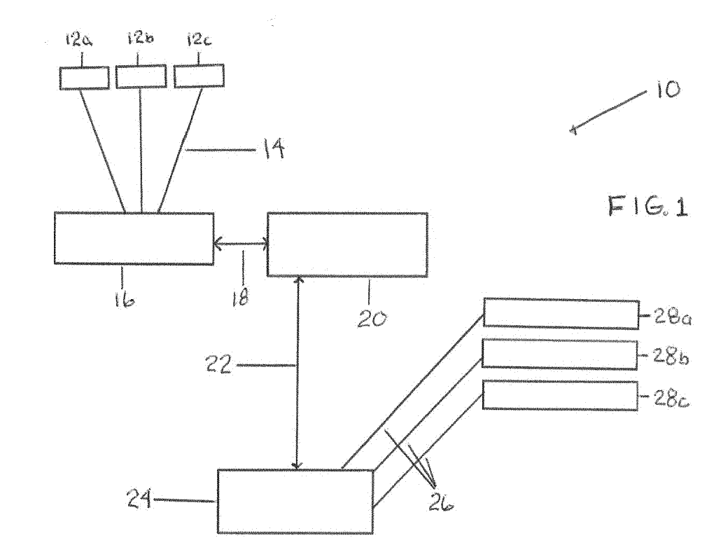

Reference to the Figures can assist in a further appreciation of the present technology and invention. FIG. 1 is a schematic of a monitoring and response system 10 according to the present invention. The system comprises positionable or wearable sensors 12a 12b 12c, signal registration/reception 16 and transmission circuitry 20, central processor and libraries in memory 28a 28b 28c for associated response/information respectively associated with the sensors 12a 12b 12c. Also shown are wireless or hardwire communication links 14 between the sensors and an optional intermediate registration/reception component 16, preferably present in the home if the component is used. This intermediate registration/reception component 16 can have security functionality also to limit access to transmitted information, identify individual patients in encrypted formats or with secure identification codes. Sensed data is transmitted (and possibly queried and sensors interrogated by a central transmission circuitry 20, again possibly having a high security functionality) through communication link 18. The central transmission circuitry 20 may be a part of or in communication with a central computing function 24 as through information pathway 24. The central computing function 24 relays sensed data from sensors 12a 12b 12c to respective memories/libraries/logic systems/processors 28a 28b 28c, so that an appropriate analysis is made of the data by a correct system, and a correct response is retransmitted through the linked system (e.g., 28a 28b 28c back through 26 to 24 to 20 (through 22), and back to 16 where information would be displayed to the patient associated with the sensors 12a 12b 12c. The central processor 24 may also assure that individual patient data associated with each unique patient is part of the transmission to the respective memories/libraries/logic systems/processors 28a 28b 28c, so that an individualized response and critical individual patient parameters is considered.

The personal health care component of the inventive integrated electronic community medical marijuana network preferably comprises a customized medical device temporarily attached to or worn by the medical marijuana patient linked (wireless, hardwired, or through a downloadable memory device that can be placed into communication to another memory, I/O port or computer) to a computer for receiving, storing, processing, and transmitting information, and a plurality of interfacing ports adapted to accept a plurality of different medical marijuana patients monitoring modules. The computer may have multiple I/O lines or bandwidth so that data flow from multiple monitoring modules can be simultaneously received and is electronically interconnected to the data processor for sending and receiving information. The data processor includes means for providing operating instructions to the monitoring modules, accessory modules, and therapy-providing modules. Each sensor module provides information on a condition of a medical marijuana patient. The data processor monitors the information provided by the modules.

The sensor device worn by a medical marijuana patient is customized to detect specific psychological, physiological and metabolic markers and parameters indicating symptoms of a particular disorder or disease for which said patient is undergoing treatment with a specific medical marijuana protocol. The psychological, physiological and metabolic parameters that are detected by the device can be processed and/or displayed immediately to the patient or caregiver, to the central computer, or to the local computer, and medical alert indications displayed when a significant abnormal parameter value is obtained. The information can also be wirelessly transmitted to the medical marijuana patient's smart phone or computer, or the smart phone or computer of the medical marijuana patient's health care provider, for storage or analysis, or transferred from a port on the monitoring module device to a flash drive or other storage medium. In a particularly preferred embodiment of the invention, a smart phone is used as a gateway to relay medical marijuana patient health data to a remote database via the mobile network, which provide remote diagnoses that enable wireless communications among mobile users, medical professionals and other healthcare care-givers in an easy, secure and efficient manner. In another preferred embodiment of the invention, the device software is based on the commercially-available cross-platform Java system and provides the user with an easy-to-use graphical user interface on their smart phone that uses the standard navigation buttons on mobile devices. The information can also be electronically transmitted to the medical marijuana health management network in which the medical marijuana user is preferably a registered patient.

The device also preferably inputs specific symptoms, frequency of symptoms, and clinical history relating to disorder and disease indicators that have been identified for treatment with medical marijuana. The recorded information can be summarized and displayed for the medical marijuana patient or his health care provider, and the patient's symptoms can be tracked, in order to encourage compliance with a prescribed medical marijuana protocol that has been identified for sensed parameters or conditions that warrant or require a corrective medical response. If the patient's symptoms improve as a result of the medical marijuana protocol, this information can be tracked over time by the medical marijuana health management network.

The medical monitoring device provides for unobtrusive use by the medical marijuana patient. Preferably, the display is only viewable on activation by the patient to prevent unintended viewers of the device display. The data can be viewed in real time or logged based on the temporal relationship to activities, or as averages, or graphic displays. The device may also be embedded in existing sports monitoring systems, audio systems and the like which are already worn in public. For example, the device may be embedded in an iPod® music storage system or an iPod® system case. It may also be as unobtrusive as a wristband (appearing as if it were a watch), GPS golf monitoring watch, tendon support device, and the like.

In addition to providing a record and display of psychological, physiological and metabolic parameters, the medical device according to the invention is also useful in monitoring disorders and diseases for which the patient is undergoing medical marijuana treatment, for example, pain caused by multiple sclerosis or spinal cord damage, nausea resulting from cancer chemotherapy, hepatitis C medications and HIV/AIDS treatment, anxiety resulting from post-traumatic stress disorder, and inflammation associated with colitis and Crohn's disease.

Disease progression, symptoms, and long term health course can preferably be monitored and recorded with the device in relation to the patient's compliance with his medical marijuana protocol. The monitoring module device may also have patient user input capability for symptoms or events that cannot be directly monitored by a particular device. The monitoring module device may, by way of non-limiting examples, have one or more specific user input capabilities (buttons, touch-screens, sliding elements, winding posts, etc.) which can be used to input subjective reports by the medical marijuana patient of dizziness, lightheadedness, visual impairment, tinnitus, mouth dryness, spasms, disorientation, memory events, sensory impairment generally, and even degrees of these events. A single input element, such as a single button, can be programmed so that specific numbers of depressions of the button within a time frame (intervals between depressions of less than 3 second, for example) can indicate different user input of conditions. For example, a single press of a button could indicate dizziness, two presses in succession indicating mouth dryness, and three presses indicate a memory event. In one preferred embodiment of the invention, the medical device provides a user interface with both voice recognition and tap-a-button user capability.

The device, through the use of software, would have the ability to alter and adapt the input screen, to incorporate new insights and findings, such as prevalence of certain symptoms or presence of symptoms not previously encoded, as the system learns from gathering data across the entire population.

In another preferred embodiment of the present invention, the medical device provides an electronic medical journal separate from records maintained by existing medical marijuana organizations, such as MJ Freeway, said medical journal record-keeping including maintaining lists of current medications, updated over time, including measuring the changes in medications over time, such as a reduction in dose or elimination of medication relative to medical marijuana use. The electronic medical journal provides a medical profile of the medical marijuana patient, including a patient's medical record with existing conditions, list of medications, including monitoring changes of medications over time with use of medical marijuana.

One exemplary type of disclosed device that may be a component of the present system is the MedWand™ device that is advertised as a device (that separates into functional components) that can separately have a patient perform diagnostic tests and forward the data from those tests to a medical analyst. The test enabled by the MedWand™ device may include temeparte?, pulse, heart rate, heart sounds, lung sounds during breathing, nasal visual inspection, eye visual inspection, throat visual inspection, skin visual inspection, oxygen blood level measurement, and EKG testing data. The intended use of the device is for the patient to self-perform the individual tests, report them to a medical diagnostician, and then have the medical diagnostician evaluate the actual data transmitted.

A further exemplary type of disclosed device is the BioStampRC wearable device made by MC10, that employs stretchable electronics. Another exemplary device is the Skin Patch device also made by MC10 that employs epidermal mechano-acoustic sensing electronics to listen to sounds created by internal organs in the human body. A further exemplary health monitor is a contact-free wireless health and sleep tracker monitor made by EarlySense that can monitor vital physiologic information, such as heart rate, body temperature, blood pressure and respiration, while the patient sleeps and alerts the patient to any abnormality.

In a particularly preferred embodiment of the invention, the medical device can record the amount, type and route of administration of medical marijuana used by the medical marijuana patient, including re-doses of medical marijuana. Although medical marijuana potency can vary between brands, lots and with age, data can also be provided into the system of the brand and lot and sales date (which is combined with use date), and with some foreknowledge of the brand and lot, an estimate of the specific therapeutic potency of the medication, along with the known body weight of the medical marijuana patients, can be used to help quantify dosage impact and immediate levels of blood content of marijuana metabolites for a specific patient taking a specific dose of a specific medical marijuana product. In a preferred embodiment of the invention, the wearable sensor device uses GPS to map geographic location and other information related to the movement of the medical marijuana patient over time, and correlates said information with the time, amount, and last dose of medical marijuana used by the patient.

In another preferred embodiment of the invention, the sensor device monitors the sleep state of the medical marijuana patient, and correlates changes in the sleep state of the patient with measured psychological, physiological and metabolic parameters and with the time, amount, and last dose of medical marijuana used by the patient.

The wireless feature of the device allows monitoring of medical marijuana patients remotely in a variety of settings, including a health care setting, such as a clinic or hospital, or at the medical marijuana patient's place of work or home. The wireless feature with the psychological, physiological and metabolic markers and indicators also permits the medical marijuana patient to be alerted or instructed to take medical marijuana in accordance with a prescribed medical marijuana protocol to alleviate particular symptoms of a particular disorder or disease. A decreased incidence of psychological, physiological or metabolic symptoms can also be tracked and recorded using the sensor device, thereby empowering medical marijuana patients to effectively and safely choose and consume the right dose and variety of marijuana customized to each individual's patient's disorder or disease.

According to the invention, the medical device could be leased or sold to medical marijuana patients, or provided through their health insurer based on the subscription agreement of the medical marijuana subscriber with a health insurance plan.

In addition to recording physiologic parameters and dosing information, the networked system will have the capability to record the cost of medications, encounters with healthcare professionals, and the purchase or acquisition of medical marijuana. This cost information will be recorded by the system and analyzed to offer retrospective and predictive insights to change in costs to patients and the system as a whole as patients begin to employ medical marijuana as part of their personal health maintenance regimen.

In conjunction with the personal health care system, the present invention provides a networked health care and monitoring system capable of providing updated reliable vital information on the health condition of medical marijuana consumers adapted to support home healthcare maintenance. The ability to monitor the health condition is linked to the recording capability for monitoring the safety and efficacy of marijuana following administration, thereby providing insight into the effects and benefits of medical marijuana. The system includes testing and measuring instruments adapted to monitor the patient's vital psychologic, physiologic, and metabolic information. The system may further include control devices having health care and maintenance functions monitored by the testing and measuring instruments in the system.

In the method of the invention, physicians develop protocols for treating patients with medical marijuana based on published guidelines for medical care, including: A patient history and detailed examination of the patient; A treatment plan with objectives; Informed consent, including discussion of side effects; Regular review of the treatment's efficacy; Consultations, as necessary; and, Detailed ongoing records supporting the decision to recommend the use of medical marijuana. The decision to use medical marijuana is based on careful assessment of the patient's condition with consideration for other possible treatments. Treatment decision-trees are routinely used.

Further in the method of the invention, a number of areas of health and illness are emphasized, such as management of chronic diseases, including prevention of acute exacerbations, prevention of acute complications, and adjustments in life-style to better live with chronic diseases, such as cancer, epilepsy and stress.

One additional feature that should be incorporated into the system and process is security with respect to personal identification of the actual patient user. This can be easily incorporated using the existing proposed equipment, which is highly significant. Where visual observation devices (skin viewing, nasal viewing, eye scanning and the like is used, these are little more than cameras. By using these same cameras as biometric data collectors, a patient may be required to show a full frontal face shot and then continuously move the view along the body of that patient for the next examination/sensing procedure, whether or not the camera is used in that next sensing device. By having the continuous view from face to contacted body part, the device cannot be transferred from the first identified patient to a substitute patient. In this way, false signal content cannot be fraudulently introduced. Also, the camera may be similarly used to read fingerprint data or retinal data and then attach the device to the hand or arm for blood reading (pulse, pressure, oxygen content), temperature taking, skin tone measurements and the like.

In one preferred embodiment of the invention, wherein the system is arranged in the centralized network configuration, the testing and measuring instruments and the control devices are connected via a local area network with a data controller, wherein all the vital information obtained in the system is stored. Instruments and devices are permitted to access the controller through the network to retrieve necessary vital information. In another preferred embodiment arranged in the distributed network configuration, the vital information obtained by respective measuring instruments is stored therein.

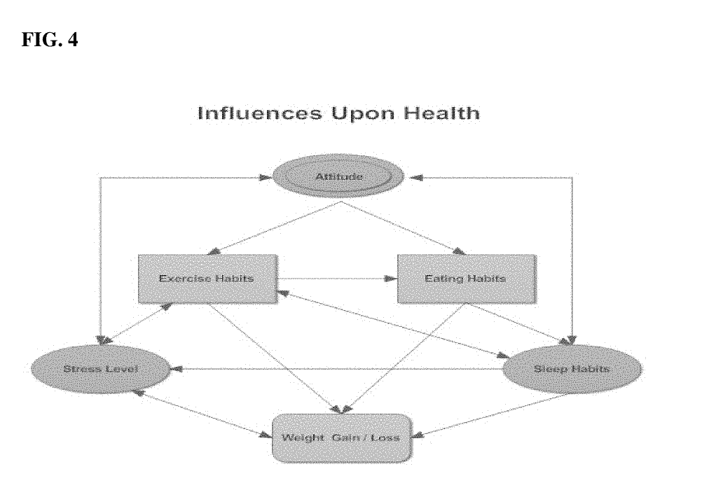

In another preferred embodiment of the invention, the approach to interpreting the imprecise (“fuzzy”) data collected by the medical device worn by the medical marijuana patient employs the use of statistical methods via influence diagrams, such as shown in the influence diagram of FIG. 4.

In conjunction with the personal health care and the networked health care and monitoring system of the present invention, a software-based medical information system performs a method of analyzing health care claims records for an enrolled population of medical marijuana consumers to assess and report on quality of care based on published and well-established quality indicators. The system analyzes health care received by medical marijuana enrollees having a specified health care condition by: providing to the system health care claims records for a selected enrollee population; defining at least one health care condition in terms of health care events reportable in health care claims records; identifying in the health care claims records those enrollees meeting the definition for that health care condition; defining health care quality criteria for that health care condition in terms of health care events reportable in health care claims records; comparing the health care quality criteria for the at least one health care condition to the health care claims records for at least a portion of those enrollees meeting the definition for that health care condition; and developing and outputting from the system a health care quality report based on the comparison and formulating action recommendations to improve care. Overall, the system provides an efficient means to supplement claims data with data from patient medical records.

In the method of the invention, the electronic personal medical marijuana health management program uses mediums available through information technology and telecommunications to provide a clinical program that promotes preventative health and wellness, emphasizing the healthy individual, responsibility for self-health management and access to health information resources. Several health information components are integral to the method and the practice of the invention: the collection of health information databases, including disease description, risk factors, and disease management; on-line health care experts; and, strong involvement of local hospitals and clinician groups to reinforce the position of local health care providers.

The first information resource available within the electronic health care clinic of the present invention—health information databases—are divided into the broad categories of people, references, places and lifestyles, which represent a cross section of local, national, and international sources that the medical marijuana consumer will search.

The second information resource within the electronic health care clinic of the present invention—on-line health care experts—covers a wide range of medical specialties available to medical marijuana consumers as they search for a personal response to a medical question through the Internet and are connected into local clinicians in the program.

In the method of the invention, email connections are enabled between clinicians and consumers to provide personal requests to the local database; to an electronic bulletin board of important announcements and health alerts of immediate concern posted by local, national, or international groups;

or to electronic chat rooms for clinicians and consumers to form support networks for specific diseases or plan local health care activities.

Still further in the method of the invention, the conceptual clinical framework for the electronic personal medical marijuana health management network includes a matrix of clinical sub-programs using dimensions of wellness or condition assessment and level of access to electronic information resources or direct contact with a health care provider, said clinical sub-program framework including, wellness programs, primary disease prevention programs, self-care management programs including risk factor databases and self-care algorithms, acute disease self-care management programs including self-care algorithms and direct contact with health care personnel, and chronic disease self-care management programs including self-care algorithms and direct contact with health care personnel

In one embodiment of the invention, the wireless network includes a satellite system. Internet includes but is not limited to intranets, local area networks and wide area networks. Computers include but are not limited to personal computers, stand-alone computers, tower computers, servers, desktop computers, laptop computers, notebook computers, personal digital assistants, work stations, main frames, minicomputers, supercomputers and wearable computers. Computer can also be a special purpose computer programmed to perform the disclosed algorithms. Wireless device includes but is not limited to cell phones, personal digital assistants, wireless Internet cards, wireless modems and smart cards. Databases include but are not limited to relational databases, object databases and post-relational databases. According to one embodiment of the invention, a computer and a database can be coupled together via an Ethernet connection that can be placed in a location such as but not limited to a government facility, a private company facility, a clinic, a vehicle or the like. Medical marijuana health care providers include but are not limited to physicians, nurses, clinic workers, pharmacists, private citizens, members of government, members of for-profit and non-profit organizations.

An integrated services digital network connection, a wireless connection, a satellite dish, or the like, may also provide the computer with a connection to the Internet according to an embodiment of the present invention. An Internet connection may also allow the computer to download data files, audio files, application program files and computer-executable method steps according to another embodiment of the present invention. The computer may also include a wireless modem and/or connection to a satellite dish for access to a wireless network. The invention is not limited to a particular number of computers or wireless devices. Any number of computers or wireless devices that can be connected to a network, such as a wireless network or the Internet, or any other network, may be used.

As indicated earlier, although applicable or wearable sensors or sensor systems are preferred, these may be used in combination with medically administered and/or medically analyzed treatments that are more invasive and may require some tissue sampling. The samples may (less preferred) be provided by the patient and transferred to the human diagnostician, or the patient will be visited by or visit and medical technologist that takes the samples, and transfers them to a tissue/DNA/RNA/telomere analyst to have those results combined with the data accumulated from the applicable/wearable device to provide more detail in data that can be used for treatment evaluation. These submicroscopic data points are becoming more important as not only can changes be observed in the tissue/DNA/RNA/telomere analysis, but also identification of pre-existing characteristic in RNA and DNA and telomeres can be used to predict results based on predetermined genetic factors.

FIG. 1 as previously explained is a flow chart illustrating one embodiment of a method of the invention for verifying a medical marijuana patient's personal information over a network. According to the invention, information can be transmitted by the medical marijuana patient or a healthcare professional, or it can be automated.

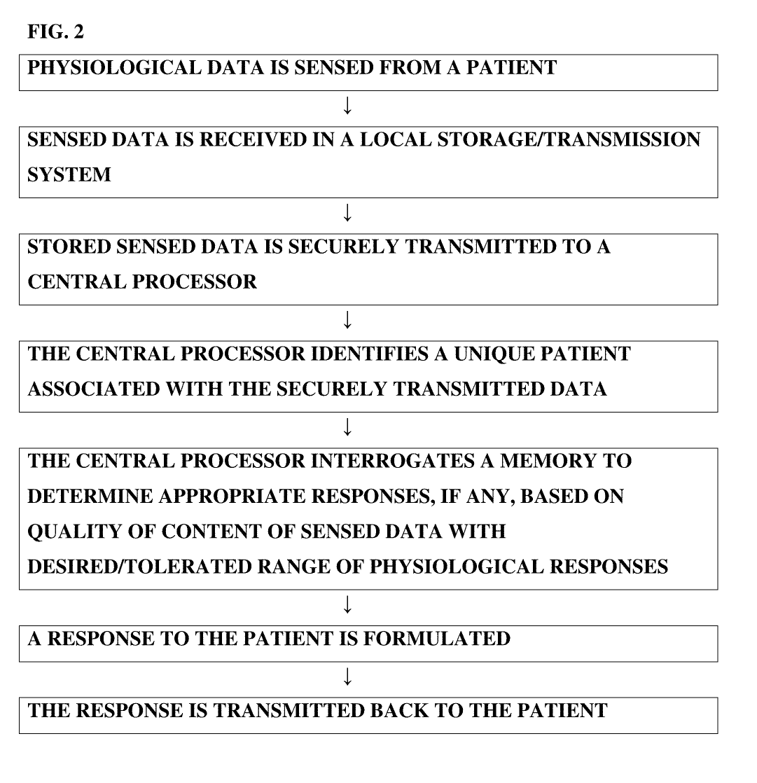

FIG. 2 is a simplified flow-chart illustrating a process for monitoring and responding to sensed psychological, physiological or metabolic markers or parameters occurring during the use of medical marijuana by a medical marijuana consumer.

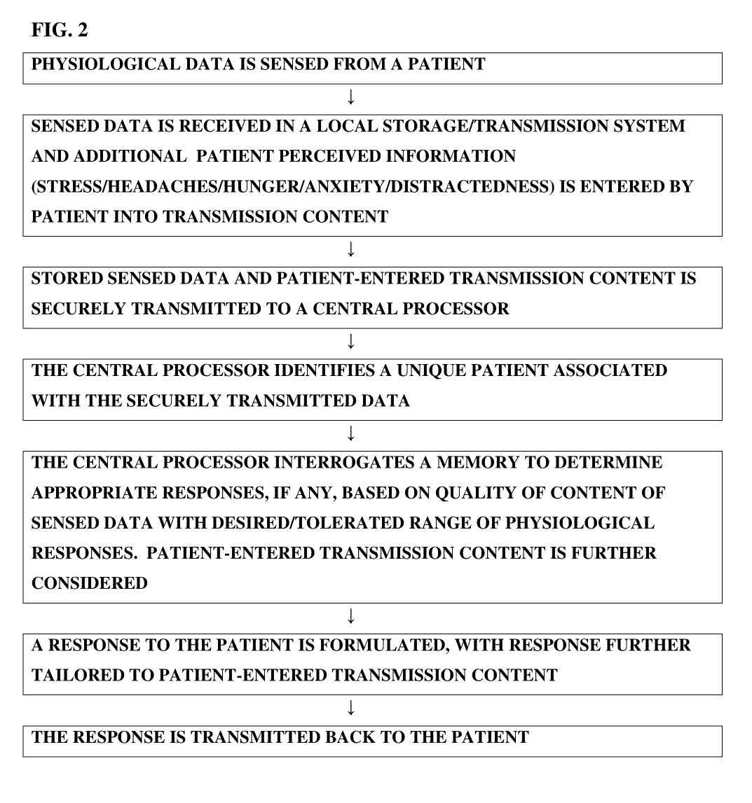

FIG. 3 is a more complex flow-chart illustrating a method for monitoring, registering and analyzing the sensed psychological, physiological and metabolic markers or parameters according to the invention. Statistical methods are employed to check the significance of changes in the sensed psychological, physiological, and metabolic markers or parameters.

The Internet connects computers worldwide through well-known protocols, such as the Transmission Control Protocol or Internet Protocol, into a vast network. Information on the Internet is stored worldwide as computer files, mostly written in the Hypertext Mark Up Language. The collection of all such publicly available computer files comprises the World Wide Web, which is a multimedia-enabled hypertext system used for navigating the Internet made up web pages, which can be displayed on a computer monitor. Each web page can have connections to other pages, which may be located on any computer connected to the Internet.

A typical Internet user uses a client program called a “Web Browser” to connect to the Internet. A user can connect to the Internet via a proprietary network, or via an Internet Service Provider. The web browser may run on any computer connected to the Internet. The Web Browser receives and sends requests to a web server and acquires information from the WWW. A web server, upon receipt of a request, sends the requested data to the requesting user. A standard naming convention Uniform Resource Locator (“URL”) has been adopted to represent hypermedia links and links to network services. Most files or services can be represented with a URL.

According to a specific embodiment of this invention, information provided by a medical marijuana patient is stored in a database, such information including the user's personal information, medical ailments, medical records, prior usage of marijuana, driver's license, age, location of domicile and government identification. According to a specific embodiment of the invention, the review of a medical marijuana user's medical status is performed by a physician preferably registered with the electronic community medical marijuana network. According to a further embodiment of the present invention, the medical marijuana patient may connect to the physician over the network, phone, or video-chat for a live medical analysis. According to another embodiment of the invention, a physician may review an application to purchase medical marijuana via in-person interview or videoconferencing. In a further embodiment of this invention, a physician is required to sign the users medical records and application for medical marijuana. According to a specific embodiment of the present invention, the physician's signature will be stored in the database and attached to the medical marijuana patient's documents.

A further embodiment of the present invention provides for insurance coverage to allow medical marijuana patients to enter their insurance information over the electronic medical marijuana network. According to this embodiment of the invention, a medical marijuana patient will enter via the internet or a wireless device his or her insurance provider's name, phone number, the insurance card holder's name, the insurance card holder's phone number, the insurance member's ID number and/or cardholders date of birth and this information will be held in a database available for future reference. According to another embodiment of the invention, the electronic medical marijuana network will track in a database for all medical marijuana patients who are members of the network.

A method for generating a medical marijuana provider profile may use a computer system, wherein said system contains comprehensive data sets comprising multiple codes selected from the group consisting of diagnostic codes, circumstance codes, preventative codes, complication codes, staging codes, and individual response codes for unique clients. Such codes can be represented as:

hospital utilization measures, including pharmacy, imaging, laboratory and supplies utilization rates; quality outcomes measurements, including readmissions rates, complication rates, and mortality rates; other performance measures, including subjective patient satisfaction; comparisons with other evidence-based medical protocols on groups or individuals; a plurality of historical medical provider patient billing records; a grouping of diagnostic codes based on individual and group average codes; a grouping of qualifying circumstance codes such as reasons for prescriptions, age, specific medical conditions; a grouping of staging indicators or base-line function analyses; a grouping of preventative codes for specific qualifying conditions and responses to treatment for those conditions and side effects; and a grouping of complication codes.

A wearable device may use an accelerometer to monitor a medical marijuana user's activity level and in reference to data sent from the wearable device into the system, a recommendation to adjust the dose of medical marijuana medication to effectively alleviate pain and reduce stress, balanced against any adverse data received through the device. The accelerometer preferably has BlueTooth® and iOS and Android® transmission capability, at least into the local server, capability with gyroscope functionality in the device, USB access, and a long-life battery. Real time data may be sent to the companion application on a smartphone.

The Bluetooth and iOS and Android compatible technology to connect to a smartphone application, wherein a user can control the device's features and track therapy. The medical device can also use cloud based technology which allows a physician to monitor a patient user's heart rate, respiration, blood pressure, oxygen, movement, and temperature in real time. Patients can monitor their medication-taking patterns on their mobile device and physicians have access to objective data that enables them to initiate, tritrate and eliminate medication.

The device is a biosensor that is reusable and has a 3-axis accelerometer that keeps track of heart rate, breathing, temperature, and can detect the body position of the user.

The device can also have a tri-axial accelerometer embedded in a smart watch style device with medical grade sensors that can monitor the position of the user and help assist them during turning motions.

The device can also be worn as a wristband or watch with embedded electrodes that are able to measure important sleep statistics. The device has a smart computer engine with sensors, speakers, and lights to monitor and stores sleep information which can be accessed from anywhere in real time.

The method may be practiced for generating a medical marijuana provider profile using a software-based medical information system that analyzes health care received by pain treatment medication enrollee programs including medical marijuana enrollees having a specified health care condition by:

providing to the system health care claims records for a selected enrollee population;

defining at least one health care condition in terms of health care events reportable in health care claims records;

identifying in the health care claims records those enrollees meeting the definition for that health care condition;

defining health care quality criteria for that health care condition in terms of health care events reportable in health care claims records;

comparing the health care quality criteria for the at least one health care condition to the health care claims records for at least a portion of those enrollees meeting the definition for that health care condition; and

developing and outputting from the system a health care quality report based on the comparison and formulating action recommendations to improve care, using a computer system,

wherein said system contains a unique client processor to access and communicate with a central processor including comprehensive data sets in the central processor comprising multiple codes selected from the group consisting of diagnostic codes, circumstance codes, preventative codes, complication codes, staging codes, and individual response codes for unique clients and defined responses in a look-up table for the unique client;

wherein the unique client communicates through the unique client processor with the look-up table for enrollees that has, for the unique client, a) input subjective reports via a voice input system of the level of pain on a per hour or more frequent basis, b) input subjective reports of at least one subjectively described condition selected from the group consisting of dizziness, lightheadedness, visual impairment, tinnitus, mouth dryness, spasms, disorientation, memory events, sensory impairment, cognitive impairment, depression, suicidal ideation, and aggression, and c) objective measurements from standardized tests providing the objective measurements selected from the group consisting of visual acuity, physical coordination based on a standardized coordination test, physical reflex results based on a standardized reflex test; and d) objective measurements that would detect and measure the concentrations of active metabolites of marijuana metabolism in the medical marijuana patient's exhaled respiratory gases; wherein a prescription for medical marijuana will be renewed for a specific enrollee when the enrollee has, over a predetermined amount of time, confirmed enrollee acceptable levels of at least three of the subjectively described conditions entered through the unique client processor and has passed at least one of the objective measurements at least three times over a 48-hour period through performing at least one standardized test in communication with the unique client processor, producing the at least one objective measurement on apparatus standardized to perform the at least one standardized test and communicate the objective measurements directly to the unique client processor with a time stamp.

The unique client generates a specific individual enrollee profile through communication from the unique client processor to the central processor based upon the at least one subjectively reported condition, and the method includes altering the medical provider profile for that individual enrollee based on the at least one subjectively reported profile, and wherein the objective measurements from standardized tests in c) are implemented through a device worn by the unique client, wherein the worn device includes an accelerometer that measures the specific individual enrollee's activity level by wireless transmission from the client processor to the central processor, the central processor referencing data stored in memory of the objective tests, comparing currently generated objective measurements with the stored data.

The worn device further includes physiological sensors and metabolic sensors and physiological data and metabolic data is sensed through the worn device to the unique client processor and to the central processor, and the central processor determines if previous medical marijuana use has resulted in symptom reduction or improved clinical outcomes for the unique individual medical marijuana patient by comparing sensed physiologic and metabolic parameters that reflect symptoms of said medical marijuana patient's disorder or disease condition, and wherein the medical marijuana unique client performs a standardized physical coordination test on a manually controlled input device selected from the group consisting of a keyboard, joy stick, optical receptor, roller ball, and mouse, wherein the manually controlled input device performs the standardized physical coordination test, and current results of the standardized physical coordination test are compared with at least one previous result from the standardized physical coordination test stored in the central processor to determine if physical coordination is within a predetermined level of tolerance of physical coordination.

The medical marijuana patient's personal information and sensed data and standardized physical coordination tests are transmitted to an electronic medical marijuana network from the unique client processor, the method comprising: receiving said medical marijuana unique patient's personal information and transmitting said medical marijuana unique patient's personal information over a network; and verifying said patient's personal information before authorizing a refill of the medical marijuana prescription.

The physiologic and metabolic parameters are transmitted to the central processor at a monitoring location in the medical marijuana health care network, with data from a wearable sensor on the unique client detecting specific psychological, physiological and metabolic markers and parameters indicating that a dose of medically prescribed marijuana has been taken.

The parameters are transmitted to a cell phone of the unique client performing as the unique client processor.

The medical marijuana unique patient is alerted of a potential health problem and the central processor advises said medical marijuana unique patient to follow a specific medical marijuana protocol which is transmitted to the unique client cell phone.

What is claimed is:

1. An integrated electronic community pain medication management health care network, comprising: a personal health care medical monitoring device configured to be in contact with a patient who is receiving medically prescribed marijuana to sense at least one psychological, physiological, or metabolic property of the patient, storage memory in communication with the medical monitoring device to store sensed data regarding the at least one psychological, physiological or metabolic property, and a software-based medical information system comprising a computer configured to receive the stored memory or through a communication link directly receive and store sensed psychological, physiological, or metabolic property data, wherein said computer is configured to receive, store, process, and transmit information, and said computer further comprises at least one of a plurality of interfacing ports, including multiplexing capability for simultaneously receiving multiple patient transmissions, and a dedicated port for a single user input wherein each said interfacing port is adapted to accept communication from a plurality of different medical monitoring devices; Wherein said computer is further configured to receive and analyze user input comprising subjective reports by a unique medical marijuana patient of at least one subjectively describable condition selected from the group consisting of dizziness, lightheadedness, visual impairment, tinnitus, mouth dryness, spasms, disorientation, memory events, sensory impairment, cognitive impairment, depression, suicidal ideation, and aggression, and the unique medical marijuana patient has sensors which sense objective measurements from standardized tests providing the objective measurements selected from the group consisting of visual acuity, physical coordination based on a standardized coordination test, physical reflex results based on a standardized reflex test, and the sensors are in communication with a unique medical marijuana patient local processor in communication with the computer.

2. The integrated electronic community pain medication management health care network of claim 1, wherein said interfacing port is in communication with the computer to enable sending and receiving information, said data computer including at least a communication network for providing operating instructions from the computer to the medical monitoring device, wherein each said network transmits information on a condition of a patient under pain medication treatment and said data processor monitors the transmitted information from the medical monitoring device.

3. The integrated electronic community pain medication management health care network of claim 1 wherein medical quality marijuana is provided by a government agency providing legal authorization for use of the medical quality marijuana during a period of time overlapping use of the medical monitoring device.

4. The integrated electronic community pain medication management health care network of claim 3, wherein said medical monitoring devices are configured to monitor, test and measure a medical marijuana patient's vital psychologic, physiologic and metabolic parameters.

5. The integrated electronic community pain medication management health care network system of claim 1, wherein instruments and devices access the computer through a network to retrieve necessary vital information, wherein the instruments and device are further configurable in the distributed network configuration to store vital information obtained by respective measuring instruments.

6. The integrated electronic community pain medication management health care network of claim 3 further comprising a software-based medical information system is configured to analyze health care claims records for an enrolled population of medical marijuana consumers to assess and report to physicians and administrators in said health care network on quality of care based on quality indicators.

7. The network of claim 6, wherein participants include at least two facilities selected from the group consisting of primary care physicians, specialist physicians, hospitals, medical marijuana laboratories, medical marijuana dispensaries, medical marijuana cultivation facilities, and medical marijuana production facilities.

8. A method for generating a medical marijuana provider profile using a software-based medical information system that analyzes health care received by pain treatment medication enrollee programs including medical marijuana enrollees having a specified health care condition by:
providing to the system health care claims records for a selected enrollee population;
defining at least one health care condition in terms of health care events reportable in health care claims records;
identifying in the health care claims records those enrollees meeting the definition for that health care condition;
defining health care quality criteria for that health care condition in terms of health care events reportable in health care claims records;
comparing the health care quality criteria for the at least one health care condition to the health care claims records for at least a portion of those enrollees meeting the definition for that health care condition; and
developing and outputting from the system a health care quality report based on the comparison and formulating action recommendations to improve care, using a computer system,
wherein said system contains a unique client processor to access and communicate with a central processor including comprehensive data sets in the central processor comprising multiple codes selected from the group consisting of diagnostic codes, circumstance codes, preventative codes, complication codes, staging codes, and individual response codes for unique clients and defined responses in a look-up table for the unique client;
wherein the unique client communicates through the unique client processor with the look-up table for enrollees that has, for the unique client, a) input subjective reports via a voice input system of the level of pain on a per hour or more frequent basis, b) input subjective reports of at least one subjectively described condition selected from the group consisting of dizziness, lightheadedness, visual impairment, tinnitus, mouth dryness, spasms, disorientation, memory events, sensory impairment, cognitive impairment, depression, suicidal ideation, and aggression, and c) objective measurements from standardized tests providing the objective measurements selected from the group consisting of visual acuity, physical coordination based on a standardized coordination test, physical reflex results based on a standardized reflex test; and d) objective measurements that would detect and measure the concentrations of active metabolites of marijuana metabolism in the medical marijuana patient's exhaled respiratory gases;
wherein a prescription for medical marijuana will be renewed for a specific enrollee when the enrollee has, over a predetermined amount of time, confirmed enrollee acceptable levels of at least three of the subjectively described conditions entered through the unique client processor and has passed at least one of the objective measurements at least three times over a 48-hour period through performing at least one standardized test in communication with the unique client processor, producing the at least one objective measurement on apparatus standardized to perform the at least one standardized test and communicate the objective measurements directly to the unique client processor with a time stamp.

9. The method of claim 8, wherein the unique client generates a specific individual enrollee profile through communication from the unique client processor to the central processor based upon the at least one subjectively reported condition, and the method includes altering the medical provider profile for that individual enrollee based on the at least one subjectively reported profile, and wherein the objective measurements from standardized tests in c) are implemented through a device worn by the unique client, wherein the worn device includes an accelcrometer that measures the specific individual enrollee's activity level by wireless transmission from the client processor to the central processor, the central processor referencing data stored in memory of the objective tests, comparing currently generated objective measurements with the stored data.

10. A method according to claim 8 wherein the worn device further includes physiological sensors and metabolic sensors and physiological data and metabolic data is sensed through the worn device to the unique client processor and to the central processor, and the central processor determines if previous medical marijuana use has resulted in symptom reduction or improved clinical outcomes for the unique individual medical marijuana patient by comparing sensed physiologic and metabolic parameters that reflect symptoms of said medical marijuana patient's disorder or disease condition, and wherein the medical marijuana unique client performs a standardized physical coordination test on a manually controlled input device selected from the group consisting of a keyboard, joy stick, optical receptor, roller ball, and mouse, wherein the manually controlled input device performs the standardized physical coordination test, and current results of the standardized physical coordination test are compared with at least one previous result from the standardized physical coordination test stored in the central processor to determine if physical coordination is within a predetermined level of tolerance of physical coordination.

11. The method of claim 10, wherein a medical marijuana patient's personal information and sensed data and standardized physical coordination tests are transmitted to an electronic medical marijuana network from the unique client processor, the method comprising: receiving said medical marijuana unique patient's personal information and transmitting said medical marijuana unique patient's personal information over a network; and verifying said patient's personal information before authorizing a refill of the medical marijuana prescription.

12. The method of claim 11 wherein said physiologic and metabolic parameters are transmitted to the central processor at a monitoring location in the medical marijuana health care network, with data from a wearable sensor on the unique client detecting specific psychological, physiological and metabolic markers and parameters indicating that a dose of medically prescribed marijuana has been taken.

13. The method of claim 12 wherein said parameters are transmitted to a cell phone of the unique client performing as the unique client processor.

14. The method of claim 13 in which the medical marijuana unique patient is alerted of a potential health problem and the central processor advises said medical marijuana unique patient to follow a specific medical marijuana protocol which is transmitted to the unique client cell phone.

15. The method of claim 11, wherein the network comprises a wireless network.

16. A system for assisting in the use of medical marijuana comprising:
a) a medical monitoring sensor configured for contact with the human body;
b) the medical monitoring sensor configured to sense at least one physiological parameter associated with responses to marijuana in the human body;
c) the sensor configured to either transmit or store sensed data relating to the at least one physiological parameter;
d) a communication network configured to receive and transmit the sensed data relating to the at least one physiological parameter either by wireless communication with the sensor or physical receipt of a memory storage device containing the stored sensed data relating to the at least one physiological parameter, the communication network including a human-enabled input device allowing input of a) subjective reports via a voice input system of the level of pain on a per hour or more frequent basis, b) subject reports on at least one subjectively describable condition selected from the group consisting of dizziness, lightheadedness, visual impairment, tinnitus, mouth dryness, spasms, disorientation, memory events, sensory impairment, cognitive impairment, depression, suicidal ideation, and aggression, and c) objective measurements from standardized tests providing the objective measurements selected from the group consisting of visual acuity, physical coordination based on a standardized coordination test, physical reflex results based on a standardized reflex test, and d) objective measurements that would detect and measure the concentrations of active metabolites of marijuana metabolism in the medical marijuana patient's exhaled respiratory gases;
e) a processor in communication with the communication network and configured to receive and store in memory the subject reports of one subjectively describable condition and transmitted sensed data relating to the objective measurements;
f) the processor configured to compare the transmitted the subject reports of one subjectively describable condition and transmitted sensed data relating to the objective measurements to a table stored in memory, the table indicating ranges of acceptable, marginal and unacceptable ranges or limits with respect to the transmitted subject reports of one subjectively describable condition and the transmitted sensed data relating to the objective measurements;
g) the processor is configured to analyze the transmitted the subject reports of one subjectively describable condition and transmitted sensed data relating to the objective measurements to an algorithm for the purpose of relating the transmitted sensed data to at least one physiological parameter;
h) the processor having at least one response in memory for providing an at least one medical response to at least one unacceptable range or limit of the transmitted sensed data relating to the at least one physiological parameter; and
i) the processor configured to transmit the at least one medical response to an authorized recipient of that at least one medical response, wherein the processor is further configured to receive and analyze user input of subjective reports by a medical marijuana patient of at least one subjectively describable condition selected from the group consisting of dizziness, lightheadedness, visual impairment, tinnitus, mouth dryness, spasms, disorientation, memory events, and sensory impairment.

17. The system of claim 16 wherein the medical monitoring sensor is an electronic device configured to be temporarily secured to a human body during a sensing period of time and the medical monitoring sensor is a noninvasive sensor with no physical structure puncturing the skin, and the medical monitoring sensor senses for at least one physiological parameter selected from the group consisting of pulse, blood pressure, oxygenation level in blood, sugar level in blood, body temperature and breathing rate, and the sensor is configured to emit electromagnetic radiation and receive returned electromagnetic radiation and provide data with respect to amounts of difference in energy levels or characteristics of received electromagnetic radiation as compared to emitted electromagnetic radiation.

18. The system of claim 17 wherein the sensor is configured to sense audio information and convert the audio information into electromagnetic signals that are stored or transmitted and wherein medical quality marijuana is provided by a government agency and legal authorization is provided by the government agency for use of the medical quality marijuana, and this use occurs during a period of time overlapping use of the medical monitoring device.

19. A system for assisting in the use of a medical pain management by provision of medically prescribed marijuana into a human patient's body comprising:
a) a medical monitoring sensor configured for contact with the human body;
b) the medical monitoring sensor configured to sense at least one physiological parameter associated with responses to the specific drugs in the human body;
c) the sensor configured to either transmit or store sensed data relating to the at least one physiological parameter;
d) a communication network configured to receive and transmit the sensed data relating to the at least one physiological parameter either by wireless communication with the sensor or physical receipt of a memory storage device containing the stored sensed data relating to the at least one physiological parameter the communication network including a human-enabled input device allowing input of subject reports on at least one subjectively describable condition selected from the group consisting of dizziness, lightheadedness, visual impairment, tinnitus, mouth dryness, spasms, disorientation, memory events, and sensory impairment, and equipment enabling sensing of objective measurements from standardized tests, the equipment providing the objective measurements selected from the group consisting of visual acuity, physical coordination based on a standardized coordination test, physical reflex results based on a standardized reflex test;
e) a processor in communication with the communication network and configured to receive and store in memory transmitted sensed data relating to the at least one physiological parameter and the subjectively describable condition and the objective measurements provided by the equipment;
f) the processor configured to compare the transmitted sensed data relating to the at least one physiological characteristic and the objective measurements to a table stored in memory, the table indicating ranges of acceptable, marginal and unacceptable ranges or limits with respect to the transmitted sensed data relating to the at least one physiological parameter;
g) the processor being configured to analyze the transmitted sensed data and objective measurements according to an algorithm developed for that the purpose of relating the transmitted sensed data and objective measurements from the equipment to at least one physiological parameter;
h) the processor having at least one response in memory for providing an at least one medical response to at least one unacceptable range or limit of the transmitted sensed data or objective measurements relating to the at least one physiological parameter; and
i) the processor configured to transmit the at least one medical response to an authorized recipient of that at least one medical response;
wherein the processor is further configured to receive and analyze user input of subjective reports by a medical marijuana patient of at least one subjectively describable condition selected from the group consisting of dizziness, lightheadedness, visual impairment, tinnitus, mouth dryness, spasms, disorientation, memory events and sensory impairment, and the objective measurements selected from the group consisting of visual acuity, physical coordination based on a standardized coordination test, physical reflex results based on a standardized reflex test.

20. The system of claim 19 wherein the equipment is associated with a visual sensor which captures a visual image and sends sensed image data to the processor, the processor configured to use at least a first configuration of a visual image as a biometric identification of a specific patient and then receive a continual stream of visual information between the first configuration and a final configuration where the medical monitoring sensor contacts the patient for sensing of a physiological parameter, and the medical monitoring sensor is an electronic device configured to be temporarily secured to a human body during a sensing period of time, with data from a wearable sensor detecting specific psychological, physiological and metabolic markers and parameters indicating that a dose of medically prescribed marijuana has been taken, and wherein the medical monitoring sensor comprises at least a three-directional accelerometer associated with an arm of the patient that senses movement of the arm of the patient and transmits sensed movement to the processor.
